(19) United States
(12) Patent Application Publication
(10) Pub. No.: US 2005/0197305 A1
(43) Pub. Date: Sep. 8, 2005

(54) Methods and compositions for blocking progression of a disease state

(76) Inventors: Martin J. Lopez, Vancouver (CA); Martin Munzer, Parkland, FL (US); Ramon Eritja, Barcelona (ES)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/917,778

(22) Filed: Aug. 13, 2004

Related U.S. Application Data
(60) Provisional application No. 60/494,854, filed on Aug. 13, 2003.

(51) Int. Cl. C12Q 1/70; C12Q 1/68; C07H 21/02; A61K 48/00

(52) U.S. Cl. 514/44; 536/23.1; 435/6; 435/5

(57) ABSTRACT

The invention includes a reverse polarity 8-aminopurine substituted oligonucleotide hairpin (parallel-stranded hairpin) referred to herein as RP8AP hairpin. Application of the invention to a biological system in a disease state results in the possible interference with a disease causing agent in a number of potential ways, for example, through interference with DNA replication, thereby preventing production of virulent pathogens (for example virus and bacteria) by inhibition of DNA synthesis, through interference with DNA transcription, including by inhibition of production of critical mRNA transcripts necessary for production of proteins essential for microbial multiplication and disease expression, and through interference on the translational level, including by inactivation of synthesized mRNA transcripts used in propagation of the disease, thereby rendering them unable to be translated.

This application claims the benefit of priority of co-pending prior U.S. Provisional Patent Application No. 60/494,854, filed Aug. 13, 2003, entitled “Methods And Compositions for Blocking Progression of a Disease State,” and having as common inventors Martin Lopez, Ramon Eritja, and Martin Munzer. That application is incorporated by reference as if fully rewritten herein.

### SEQUENCE IDENTIFICATION LISTING

This application includes sequence identification listings both on paper and in computer readable form. Applicants state that the sequence listing information recorded in computer readable form is identical to the written (on paper) sequence listing.

### FIELD

The present invention relates to the field of therapeutics, including in vivo inhibition of any target, such as a pathologic target. The invention includes the use of parallel stranded hairpins, including parallel stranded hairpins containing 8-aminopurine residues, and their ability to bind a target molecule of interest. The targets may include, for example, bacterial, viral, rickettsial, fungal or parasitic (all microbes) targets but are not limited to these. The invention also includes destruction of cellular targets including dysphasic cells, cancer cells, and any unwanted cell subset. The present invention mediated by three strategies can inhibit replication of any unwanted targets in vivo. A therapeutic product can be designed to inhibit a specific target and evidence little or no toxic effect on the human host. The invention includes methods and compositions using oligonucleotide structures in treatment processes designed to inhibit disease progression to allow subsequent target elimination from the host, resulting in cessation of the disease state and cure.

### BACKGROUND

Strategies used in blocking pathogen replication and continued expression of a pathologic state (including, for example, cancer) may include antisense and antigene strategies. Oligonucleotide technology includes a number of interesting strategies that entail manipulation of cellular functions. For example, synthetic oligonucleotides have been purportedly used to inhibit messenger RNA (mRNA) translation (an antisense strategy), to destroy specific mRNA molecules (a ribozyme strategy), to interfere with function of particular proteins (an aptameric strategy); or to modulate the expression of individual genes by targeting a genome (an antigene strategy); Braasch, et al., “Antisense inhibition of gene expression in cells by oligonucleotides incorporating locked nucleic acids: effect of mRNA target sequence and chimera design,” Nucleic Acids Research 2002 30: 5160-5167; Kurreck, et al., “Design of antisense oligonucleotides stabilized by locked nucleic acids,” Nucleic Acids Research 2002 30: 1911-1918; Tallet-Lopez, et al., “Antisense oligonucleotides targeted to the domain IIId of the hepatitis C virus IRES compete with 40S ribosomal subunit binding and prevent in vitro translation,” Nucleic Acids Research 2003 31: 734-742; Sullivan, et al., “Hammerhead ribozymes designed to cleave all human rod opsin mRNAs which cause autosomal dominant retinitis pigmentosa,” Molecular Vision 2002 8: 102-113; Wang, et al., “A general approach for the use of oligonucleotide effectors to regulate the catalysis of RNA-cleaving ribozymes and DNAzymes,” Nucleic Acids Research 2002 30: 1735-1742; McKay, et al., “Characterization of a potent and specific class of antisense oligonucleotide inhibitor of human Protein Kinase C-α expression,” J. Biological Chemistry 1999 274: 1715-1722; Yadava, “Nucleic Acid Therapeutics: Current Targets for Antisense Oligonucleotides and Ribozymes,” Molecular Biology Today 2000 1: 1-16; Schumacher, et al., “Exposure of human vascular smooth muscle cells to Raf-1 antisense oligodeoxynucleotides: Cellular responses and pharmacodynamic implications,” Molecular Pharmacology 1998 53: 97-104; Hicke, et al., “Tenascin-C aptamers are generated using tumor cells and purified protein,”. Journal of Biological Chemistry 2001 276:48644-48654; Rhodes, et al., “The generation and characterization of antagonist RNA aptamers to human oncostatin,” M. Journal of Biological Chemistry 2000 275: 28555-28561; Giovannangeli, et al., “Accessibility of nuclear DNA to triplex-forming oligonucleotides: The integrated HIV-1 provirus as a target,” Proc Natl Acad Sci USA 1997 94: 79-84; McGuffie, et al., “Anti gene and antiproliferative effects of a c-myc-targeting phosphorothioate triple helix-forming oligonucleotide in human leukemia cells,” Cancer Research 2000 60: 3790-3799; Zhou-Sun, et al., “A physico-chemical study of triple helix formation by an oligodeoxythymidylate with N3′->P5′ phosphoramidate linkages,” Nucleic Acid Research 1997 25: 1782-1787. FIG. 1 includes a representation of exemplary antisense strategies.

In principle, an antigene strategy may have some advantages over the others listed above, because there are fewer copies of the DNA target in the cell than there are mRNA transcripts and proteins. In such a strategy, the oligonucleotide theoretically recognizes and binds in a sequence-specific manner to the major groove of duplex DNA, where the duplex DNA shows a polypurine-polypyrimidine sequence motif. Binding is said to occur through formation of a local triple-helical complex that may inhibit the biological function encoded in the DNA region of the target. The triplex-forming oligonucleotides (TFOs) can be viewed as artificial transcription repressors, in particular when the binding site is located in critical sites within the promoter of the target gene; Kovacs, et al., “Triple helix-forming oligonucleotide corresponding to the polypyrimidine sequence in the rat alpha 1 (I) collagen promoter specifically inhibits factor binding and transcription,” Journal of Biological Chemistry 1996 271: 1805-1812.

An antisense strategy, which uses short oligonucleotides to block mRNA translation through the formation of DNA: RNA duplex hybrids, is the most advanced strategy among those using oligonucleotides; Mani, et al., “Phase I clinical and pharmacokinetic study of protein kinase C-alpha antisense oligonucleotide ISIS 3521 administered in combination with 5-fluorouracil and leucovorin in patients with advanced cancer,” Clinical Cancer Research 2002 8: 1042-1048. One concept of antisense technology includes utilization of calculated nucleic acid sequences to down-regulate specific gene expression in the cells of interest. While applicants do not wish to be bound by any particular theory, it is said that a classical antisense approach functions by interaction with a transcript of a gene, called messenger RNA (mRNA), that is implicated in disease progression and/or maintenance.

Genetic information is contained within the chemical structure of DNA. In gene expression, the information encoded in the genes allows production of specific proteins that carry out cell functions. The first step of gene expression is called transcription because the information in DNA is transcribed into the nucleotide sequence of another nucleic acid, RNA. The mRNA travels out of the nucleus and in a second step of gene expression, the information contained in the endoplasmic reticulum region of the cell cytoplasm in mRNA transcripts, directs the construction of specific proteins in the ribosomes. This process is called translation because the linear array of nucleotides in the mRNA is “translated” into a corresponding sequence of amino acids to form the protein. FIG. 2 includes a representation of cellular processing of genetic information. While DNA is found in the double helical form, RNA transcripts are usually single stranded. Because a messenger RNA (mRNA) strand is ultimately translated by the cell, it is customarily called the “sense” strand. A nucleic acid strand that is complementary to, and can hybridize with, at least part of a sense strand is called an “antisense′” strand. The hybridization of an antisense strand to a sense mRNA may interfere with translation of a protein associated with that mRNA. In theory, nucleic acids that are antisense to the RNA transcript of a gene, such as for example a gene key to or otherwise involved in pathogen replication, or a deleterious human gene like those involved in certain cancers, could impair the expression of this gene, thereby disabling the particular disease state.

An antisense oligonucleotide may be a small, chemically modified strand of DNA that is designed to be opposite (“anti”) to a coding sequence of mRNA. Faria, et al., “Phosphoramidate oligonucleotides as potent antisense molecules in cells and in vivo,” Nature Biotechnology 2001 19: 4044. Oligonucleotides as short as about 15 mer have specificity sufficient to inhibit gene expression of a particular gene by annealing to an appropriate mRNA transcript. The mode of action of an antisense oligonucleotide in cells is dependent upon its composition (sugar, backbone, and base residues), and mRNA binding site location (5′-UTR, coding region, 3′-UTR); Baker, et al., “2′-O-(2-Methoxy)ethyl-modified Anti-intercellular adhesion molecule 1 (ICAM-1) Oligonucleotides selectively increase the ICAM-1 mRNA level and inhibit formation of the ICAM-1 translation initiation complex in human umbilical vein endothelial cells,” Journal of Biological Chemistry 1997 272: 11994-12000. After hybridization of an antisense oligonucleotide to the a target, the resulting double strand structure may prevent the mRNA from being translated into proteins. FIG. 3 shows a mechanism of antisense inhibition of mRNA transcripts.

In addition to, or lieu of, inhibition of mRNA transcription, a double-stranded structure may be recognized as abnormal by the cell and may be destroyed by an enzyme, for instance, by Ribonuclease H(RNase H); Vickers, et al., “Effects on RNA secondary structure on cellular antisense activity,” Nucleic Acids Research 2000 28: 1340-1347. Concisely, when an antisense oligonucleotide binds to its complementary mRNA molecule, the result is the functional destruction of that message. FIG. 3 shows a mechanism of destruction of unwanted mRNA transcripts in cell by exemplary antisense oligomers. Problems may be encountered when oligonucleotides are used in cellular systems and in vivo. One obstacle to use of antisense oligonucleotides for therapeutic or research purposes is the selection of an appropriate target site from a given mRNA sequence.

For antisense therapy to be effective, a desired complementary target sequence should be accessible for hybridization. RNA nucleotides may be inaccessible or have a decreased accessibility, for example, when they are sequestered in a secondary and/or tertiary structure that may affect the affinity and rate of oligonucleotide hybridization. If the antisense oligonucleotide can direct RNAse H activity in vivo, then there is a potential to search for target sites along all the mRNA. However, in cases wherein oligonucleotides do not exert activity through an RNAse H-mediated mechanism, target sites on mRNA may be limited to a functional site on the 5′ untranslated region (UTR) of the RNA, such as for example the CAP site, as actively translating ribosomes have considerable ability to penetrate secondary structure; Pongracz, et al., “α-Oligodeoxyribonucleotide N3′→P5′ phosphoramidates: synthesis and duplex formation,” Nucleic Acids Research 1998 26: 1099-1106. The mechanism for which the majority of antisense oligonucleotides have been designed is translational arrest by binding to the translation initiation codon, though others may be possible.

Another factor that may influence antisense activity is the chemical stability of an oligonucleotide. An oligonucleotide used for antisense therapy should evade breakdown, for example by nucleases, which may cleave unprotected nucleic acids. Phosphodiester oligonucleotides (normal DNA) are nuclease sensitive. They are rapidly degraded in biological fluids and in cells by exo- or endo-nucleases, which hydrolyze the phosphodiester linkage; consequently, they may not be effective antisense molecules. Moreover, the nucleotide-5′ monophosphates, resulting from oligonucleotide degradation, can also negatively affect cell growth and proliferation; Vaerman, et al., “Antisense Oligodeoxyribonucleotides Suppress Hematologic Cell Growth Through Stepwise Release of Deoxyribonucleotides,” Blood 1997 90: 331-339. To make antisense oligonucleotides more resistant to nucleases and extend their half-life, a number of strategies have been explored. For example, based on an assumption that degradation is mainly due to exonucleases, oligonucleotides are prepared with modifications at the ends to stabilize the sequence. It has been reported that oligonucleotides modified at both 5′ and 3′ ends with inverted thymidine produce oligos resistant to nucleases; Takei, et al., “5′-, 3′-Inverted-thymidine-modified antisense oligodeoxynucleotide targeting midkine: its design and application for cancer therapy,” Journal of Biological Chemistry 2002 26: 23800-23806. Structural analogues of phosphodiester oligodeoxynucleotides, such as phosphorothioates and methylphospbonates; N3′-P5′ phosphoramidate, morpholino modified or peptide nucleic acid modified oligos (PNAs), have been also been reported to be resistant to nuclease degradation and still reportedly able to bind to mRNA targets; Tari, et al., “Cellular uptake and localization of liposomal-methylphosphonate oligodeoxynucleotides,” Journal of Molecular Medicine 1996 74: 623-628; Gryaznov, et al., “Oligonucleotide N3′ →P5′ phosphoramidates as antisense agents,” Nucleic Acids Research 1996 24: 1508-1514; Summerton, et al., “Antisense Oligomers: Design, preparation, and properties,” Antisense & Nucleic Acid Drug Development 1997 7: 187-195; Taylor, et al., “In vitro efficacy of morpholino-modified antisense oligomers directed against tumor necrosis factor-alpha mRNA,” Journal of Biological Chemistry 1996 271: 17445-17452; Knudsen, et al. “Antisense properties of duplex-and triplex-forming PNAs,” Nucleic Acids Research 1996 24: 494-500. FIG. 4 shows exemplary chemical structures utilized to render an antisense oligomer resistant to cellular nuclease attack.

Antisense oligonucleotides may also be molecules that poorly diffuse across the cell membrane due to their ionic character. Naked nucleic acids have a strong negative charge arising from phosphate groups in their chemical backbone. This property makes such an oligonucleotide more soluble in water but insoluble in lipid, and therefore unwilling to pass through the cell membrane, which is a lipid bilayer. Within a cell, antisense oligonucleotides are reportedly trapped in endosomes and trafficked through endocytic pathway. Only a small portion escapes to the cytosol and most of them are degraded in the lysosomes.

Different strategies have been reported to increase intracellular penetration and the cytoplasmic release of an antisense oligonucleotide. Cationic lipids in the form of liposomes, and nanoparticles have been reported to be efficient carriers for antisense oligonucleotide delivery into cells; Zelphati, et al., “Mechanism of oligonucleotide release from cationic liposomes,” Proc Natl Acad Sci USA 1996 93: 11493-11498; Junghans, et al., “Antisense delivery using protamine-oligonucleotide particles,” Nucleic Acids Research 2000 28:E45. When oligonucleotides are mixed with cationic lipids they may form a condensed and tight structure. In such a structure a liposome cannot fuse directly with the cell membrane and must be endocytosed. However, once internalized the liposome may cause a disruption of the endosomal membrane, resulting in fusion and expulsion of its contents into the cytoplasm. The use of immunoliposomes may allow cell-specific delivery of the antisense; Davis, et al., “Drug delivery systems based on sugar-macromolecules conjugates,” Current Opinion in Drug Discovery & Development 2002 5: 279-288; Huwyler, et al., “Brain drug delivery of small molecules using immunoliposomes,” Proc Natl Acad Sci USA 1996 93: 14164-14169; Lewis, et al., “A serum-resistant cytofectin for cellular delivery of antisense oligodeoxynucleotides and plasmid DNA,” Proc Natl Acad Sci USA 1996 93: 3176-3181.

Another strategy reportedly used for cellular delivery of antisense oligonucleotides is to link antisense oligonucleotides to proteins and peptides that have the ability to penetrate the cell membrane without the liposome complex; Morris, et al., “A new peptide vector for efficient delivery of oligonucleotides into mammalian cells,” Nucleic Acids Research 1997 25: 2730-2736. If the antisense needs to be translocated to the nucleus, a nuclear localization sequence (NLS) may reportedly be added to the protein sequence; Penco, et al., “Identification of an import signal for, and nuclear localization of human lactoferrin,” Biotechnol Appl Biochem 2001 34: 151-159.

Some desirable features of an antisense oligonucleotide may include, for example, strong and specific binding to the target RNA strand, resistance to nucleolytic degradation, effective cellular penetration and rapid cleavage of antisense-RNA hybrid by cellular RNAse H.

Exploitation of antisense oligonucleotide technology for the development of rationally designed therapeutic drugs for cancer chemotherapy or other diseases may rely on pharmacokinetic and toxicological properties of the selected antisense oligonucleotides. In a therapeutic context, to get a higher therapeutic specificity, the ability of an oligonucleotide to bind selectively to specific sequences in the nucleic acid targets is an important but not determinative factor. One of the causes of toxicity is related to specificity (antisense-specific toxicity). Toxicity could arise due to hybridization of an antisense oligonucleotide to an undesired target. While some studies have reported that specificity can be achieved by an antisense oligonucleotide targeted to a single base point mutation, other studies have suggested that some forms of antisense may affect both specific target mRNA as well as irrelevant (non-specific target) mRNAs; Monia, et al., “Selective inhibition of mutant Ha-ras mRNA expression by antisense oligonucleotides,” Journal of Biological Chemistry 1992 267: 19954-19962; Fisher, et al., “Evaluating the Specificity of Antisense Oligonucleotide Conjugates: A DNA Array Analysis,” Journal of Biological Chemistry 2002 277: 22980-22984. Since in most cases an antisense mechanism of action includes reliance on RNAse H activity, and as little as a 5-base complementary region of an antisense oligonucleotide to its target is sufficient to elicit RNAse H activity, binding of an antisense oligonucleotide to a non-specific RNA due to partial sequences matches could result in scission, that is, irrelevant cleavage, of those mRNAs; Ma, et al., “Intracellular mRNA cleavage induced through activation of RNAse P by nuclease-resistant external guide sequences,” Nature Biotechnology 18: 58-61.

Antisense oligonucleotides may also have biological effects unrelated to specific degradation or blockade of the target RNA. These may be referred to as hybridization-independent toxicities. These may result from non-specific interaction of an antisense oligonucleotide with proteins, or related to the presence of characteristic nucleotide motifs on the oligonucleotide which are contained with higher frequency on DNA different from vertebrates; Summerton, et al., “Morpholino and Phosphorothioate Antisense Oligomers compared in Cell-Free and In-Cell Systems,” Antisense & Nucleic Acid Drug Development 1997 7: 63-70; Weiner, et al., “Immunostimulatory oligodeoxynucleotides containing the CpG motif are effective as immune adjuvants in tumor,” Proc Natl Acad Sci USA 1997 94: 10833-10837.

It has been reported that there is usefulness in in vivo antisense therapy for modified oligonucleotides like charged phosphorothioate oligo-deoxynucleotides (PS-ODN); Mani, et al., “Phase I clinical and pharmacokinetic study of protein kinase C-alpha antisense oligonucleotide ISIS 3521 administered in combination with 5-fluorouracil and leucovorin in patients with advanced cancer,” Clinical Cancer Research 2002 8: 1042-1048; Yuen, et al., “Clinical studies of antisense therapy in Cancer,” Frontiers in Bioscience 2000 d588-593; Yuen, et al., “Phase I study of an Antisense Oligonucleotide to Protein Kinase C-α (ISIS 3521/CGP 64128A) in Patients with Cancer,” Clinical Cancer Research 1999 5: 3357-3363; however, PS-ODN are said to generate a plethora of nonantisense effects due at least in part to interactions with extracellular and cellular proteins; Anselmet, et al., “Non-Antisense cellular responses to oligonucleotides,” FEBS Letters 2002 510: 175-180. Intravenous infusion of PS-ODN in monkeys is reported to activate a complement cascade, and is said to produce transient marked fluctuations in peripheral total white blood cells and neutrophil counts, as well as a decrease in arterial blood pressure; Henry, et al., “Activation of the Alternate Pathway of Complement by a Phosphorothioate Oligonucleotide: Potential Mechanism of Action,” The Journal of Pharmacology and Experimental Therapeutics 1997 281: 810-816. A transient inhibition of clotting times was said to be due to PS-ODN in monkeys and humans; Sheehan, et al., “Phosphorothioate Oligonucleotides inhibit the Intrinsic Tenase Complex,” Blood 1998 92: 1617-1625. Those side effects are independent of the sequence of the PS-ODN but intrinsic to its chemical structure. PS-ODN side effects observed in clinical studies performed in humans include fatigue, fever, thrombocytopenia, nausea, rash, and complement activation.

Antisense oligonucleotides containing unmethylated CpG motifs (Cytosine-phosphate-Guanine) are also said to activate host defense; Carson, et al., “Oligonucleotide Adjuvants for T Helper 1 (Th1)-specific Vaccination,” J Exp Med 1997 186: 1621-1622. Because unmethylated CpG motifs are prevalent in bacterial but not vertebrate genomic DNA, this kind of oligonucleotide may be recognized as foreign DNA. Therefore, antisense oligonucleotides that resemble sequences found in bacterial DNA are potent immunostimulatory agents capable of inducing a complex immune response.

Another issue that has limited practical application of an antisense approach is difficulty in delivering oligonucleotide drugs to the target tissue in amounts large enough to be efficacious but small enough to be without significant toxicity. The therapeutic potential of oligonucleotide therapy may be influenced by bioavailability of oligonucleotides to their target cells and organs.

Delivery of therapeutic antisense to target cells in vivo is a challenge of gene therapy. Local delivery represents one way to solve this problem, and its effectiveness is reported by Fomivirsen™ (ISIS 2922, Vitravene™ brand name in U.S.), a reportedly commercially available antisense oligonucleotide. EpiGenesis Pharmaceuticals, Inc. has reported development of Respirable Antisense Oligonucleotides (RASONs); Nyce, et al., “Respirable antisense oligonucleotides (RASONS): Formulation and delivery in theory and practice,” Respiratory Drug Delivery 2000 VII: 13-17; Tanaka, et al., “Respirable Antisense oligonucleotides: a new drug class for respiratory disease,” Respiratory Research 2001 2: 5-9, a class of therapeutics for the treatment of respiratory diseases. RASONs represent another attractive form of local delivery.

Although several types of antisense oligonucleotides have been studied, the most extensively studied have been antisense oligonucleotides including phosphorothioate oligodeoxynucleotides. Most of the pharmacokinetic analysis of phosphorothioate oligonucleotides has relied on radiolabel tracer analysis both preclinically and clinically; Ali, et al., J. Am J Respir Crit Care Med 2001 163: 989-993; Leeds, et al., “Pharmacokinetics of a potential human cytomegalovirus therapeutic, a phosphorothioate oligonucleotide, after intravitreal injection in the rabbit,” Drug Metabolism and Disposition 25: 921-926. Similarities may exist between sequences with regard to plasma disposition and tissue distribution of radiolabel associated with phosphorothioate oligonucleotides.

Intravenous injection of phosphorothioate oligonucleotides in animals reveals a biphasic plasma elimination, with an initial half-life of about 0.5 hours that represents distribution out of the plasma compartment. A second half-life of about 35 to about 50 hours represents elimination from the body; Geary, et al., “Antisense oligonucleotides inhibitors for the treatment of cancer: 1. Pharmacokinetic properties of phosphorothioate oligodeoxynucleotides,” Anti-Cancer Drug Design 1997 12: 383-393. The liver and kidney are the organs with highest uptake of the oligonucleotide.

Intact phosphorothioate oligonucleotides residing in tissues are slowly metabolized and exhibit slow overall clearance from tissues with reported half-lives of about one to about two days in rodents and in excess of about three days in primates. The slow clearance from tissues allows conjecture that phosphorothioate oligonucleotides would accumulate in some tissues upon repeated administration and be potentially toxic to a patient. The pharmacokinetics of phosphorothioate oligonucleotides are said to be independent of sequence and the pattern of distribution to organs is reportedly similar across species and independent of the route of administration (for instance, intravenous versus subcutaneous). The observed plasma pharmacokinetics appears to be more closely related to body weight across species than surface area. This correlation between species provides a level of confidence that pre-clinical animal models can be predictive of exposure in the clinic, and thus, exposure can be managed based on the knowledge gained in the non-clinical studies.

Another target for selective inhibition of cancer cells involves the development of oligonucleotide inhibitors of telomerase enzyme activity. Telomerase activation appears to be an event involved in malignant transformation of normal cells; Meyerson, “Role of Telomerase in Normal and Cancer cells,” Journal of Clinical Oncology 2000 18: 2626-2634; Shea-Herbert, et al. “Oligonucleotide N3′→P5′ phosphoramidates as efficient telomerase inhibitors,” Oncogene 2002 21: 638-642. Inhibition of telomerase activity may therefore result in cancer cell senescence and death. Accordingly, inhibition of telomerase activity by the use of antisense and/or inhibitor oligonucleotides is worthy of pursuit.

Telomerase is a ribonucleoprotein composed of a catalytic subunit containing conserved reverse transcriptase motifs; an RNA subunit (hTR) used as a template for the synthesis telomeric DNA; and auxiliary proteins; Bachand, et al., “Human Telomerase RNA-protein interactions,” Nucleic Acids Research 2001 29: 3385-3393; Ramakrishnan, et al., “Characterization of human telomerase complex,” Proc Natl Acad Sci USA 1997 94: 10075-10079. Assembly of telomerase occurs in the nucleolus; Pederson, “The Plurifunctional nucleolus,” Nucleic Acids Research 1998 26: 3871-3876. The enzyme is responsible mainly for the synthesis of d-(TTAGGG)n telomeric repeats (Telomeres). Telomeres are specialized nucleoprotein structures that define the ends of chromosomes and are essential for chromosomal stability. Telomerase activity is not detected in most somatic cells. Cells of constantly renewable tissues and germ line cells are exceptions. In humans, this enzyme is of medical interest due to its role in unlimited cellular proliferation, a hallmark of cancer cells.

Telomere maintenance by telomerase has been reported for about 85 to about 90% of human cancer specimens from a large of different cancer types; Matthes, et al., “Telomerase protein rather than its RNA is the target of phosphorothioate-modified oligonucleotides,” Nucleic Acids Research 1999 27: 1152-1158. The observed differences in telomerase activity in normal cells versus tumor derived cells resulted in the hypothesis that telomerase may represent a suitable target for specific anti-cancer therapies. In accordance, several classes of telomerase inhibitors were reportedly prepared and evaluated. Those potential inhibitors include, for example, small molecules like the tea catechin, the alkaloid berberine as well as berberine-like compound, compounds capable of interacting with DNA G-quadruplex secondary DNA structures, antisense RNA, and a variety of modified oligonucleotides including for example ribozymes, phosphoramidates, phosphorothioates, and methyl phosphorothioate chimera; Seimiya, et al., “Telomere shortening and growth inhibition of human cancer cells by novel synthetic telomerase inhibitors MST-312, MST-295, and MST-199,” Molecular Cancer Therapeutics 2002 1: 657-665; Naasani, et al., “FJ5002: A potent Telomerase inhibitor identified by exploiting the Disease-oriented screening program with COMPARE analysis,” Cancer Research 1999 59: 4004-4011; Read, et al., “Structure-based design of selective and potent G quadruplex-mediated telomerase inhibitors,” Proc Natl Acad Sci USA 2001 98: 48444849; Smaglik, “Turning to Telomerase: As Antisense strategies emerge, basic questions persist,” The Scientist 1999 13: 8; White, et al., “Telomerase inhibitors,” TRENDS in Biotechnology 2001 19: 114-120; Ludwig, et al., “Ribozyme cleavage of Telomerase mRNA sensitizes epithelial cells to inhibitor of Topoisomerase,” Cancer Research 61: 3053-3061; Gryaznov, et al., “Telomerase Inhibitors-Oligonucleotide phosphoramidates as potential therapeutic agents,” Nucleosides Nucleotides & Nucleic Acids 2001 20: 401-410; Pruzan, et al., “Allosteric inhibitors of telomerase: oligonucleotides N3′→P5′ phosphoramidates,” Nucleic Acids Research 2002 30: 559-568; Elayadi, et al., “Inhibition of telomerase by 2′-O-(2-methoxyethyl) RNA oligomers: effect of length, phosphorothioate substitution and time inside cells,” Nucleic Acids Research 2001 29: 1683-1689; Pitts, et al., “Inhibition of human telomerase by 2′-O-methyl-RNA,” Proc Natl Acad Sci USA 199895: 11549-11554.

It has been reported that oligonucleotides bind to homopurine-homopyrimidine sequences of double stranded DNA by forming triple helices. Güimil, R., et al. “Theoretical calculations, synthesis and base pairing properties of oligonucleotides containing 8-amino-2′-deoxyadenosine,” Nucleic Acids Res. (1999) 27: 1991-1999. One of the problems for the development of applications based on triple helix formation is the low stability of triple helices, especially at neutral pH (physiological pH). To overcome this problem effort has been directed to design and preparation of modified oligonucleotides in order to enhance triple helix stability. The most studied type of triple helix formation is the so called purine:pyrimidine:pyrimidine motif (FIG. 5). In this motif, the purine:pyrimidine strands correspond to the target double stranded DNA sequence (known as the Watson-Crick purine and pyrimidine strands), and the Hoogsteen strand is a pyrimidine strand used for the specific recognition of the double-stranded DNA, as reported in Soliva R., et al., “DNA-triplex stabilizing properties of 8-aminoguanine.” Nucleic Acids Res 2000 28: 4531-4539.

Most of the reported base analogues studied for triplex helix stabilization are modified pyrimidines located at the Hoogsteen strand. However, an alternative approach based on the use of parallel-stranded duplexes has been reported. In an exemplary parallel-stranded duplex, purine residues are linked to a pyrimidine chain of inverted polarity by 3′-3- or 5′-5′ internucleotide junctions (FIG. 6). Such parallel-stranded DNA hairpins have reportedly been synthesized and are said to bind single-stranded DNA and RNA targets by triplex formation; Kandimalla, et al., “Hoogsteen DNA duplexes of 3′-3- and 5′-5′-linked oligonucleotides and triplex formation with RNA and DNA pyrimidine single strands: experimental and molecular modeling studies,” Biochemistry 1996 35: 15332-15339. Oligonucleotides containing 8-aminopurines may replace natural purines in triplexes. The introduction of an amino group at position 8 of the adenine, guanine, and hypoxanthine, increases the stability of triplex helix owing to the combined effect of the gain in one Hoogsteen purine-pyrimidine H-bond, and the ability of the amino group to be integrated into the ‘spine of hydration’ located in the minor-major groove of the triplex structure.

### SUMMARY

Therapeutic modalities in the past have included, for example, antibiotics, antibody molecules of various specificity, and drugs that inhibit pathogen growth and/or tumor growth. Difficulties with these strategies include selection of antibiotic resistant pathogens, inability to maintain intact active antibody molecules in the circulation due to their spontaneous clearance, and drugs that evidence toxicity to the host.

What is desired is a specific, selective, and stable composition that in vivo may not only inhibit replication of pathogens but may facilitate their subsequent clearance. Also desired is a composition that that may inhibit dysplastic or cancer cells or any other unwanted cell subset in vivo, mediated by introduction of a selected oligonucleotide probe. Of course, the aspects of the invention discussed herein should not be construed to limit the invention as defined in the claims.

In one aspect, the invention includes a reverse polarity 8-aminopurine substituted oligonucleotide hairpin (parallel-stranded hairpin) referred to herein as RP8AP hairpin. Application of the invention to a biological system in a disease state results in the possible interference with a disease causing agent in a number of potential ways, for example, through interference with DNA replication, thereby preventing production of virulent pathogens (for example virus and bacteria) by inhibition of DNA synthesis, through interference with DNA transcription, including by inhibition of production of critical mRNA transcripts necessary for production of proteins essential for microbial multiplication and disease expression, and through interference on the translational level, including by inactivation of synthesized mRNA transcripts used in propagation of the disease, thereby rendering them unable to be translated. Application of an aspect of the invention may result in the direct inhibition of disease related DNA replication of a DNA of the pathologic agent or the DNA of a cancer cell. Application of a further aspect of the invention may involve direct inhibition of pathogen mRNA transcripts involved in the infection, cancer or other pathologic course in the host. A still further aspect of the invention may involve the direct inhibition of the translation of pathogen mRNA transcripts present, resulting in an inability to support pathogen replication or continued expression of a pathologic state, such as cancer in a host. In a yet further aspect, embodiments of the invention may be designed to evidence little or no toxicity to a human and/or other host.

To offer oligonucleotide therapy in vivo, RP8AP Hairpins include structures that may form a stable triplex with a single strand DNA or RNA target at physiologic pH, for example a neutral pH, or for example a pH between from about 4 to about 7.6, including in vivo, and possesses the ability to irreversibly inactivate the target function to which it is directed to inhibit, and which may provide little or no toxicity to the normal host.

In one aspect, the invention provides a method for binding a target oligonucleotide, comprising providing a target oligonucleotide, said target oligonucleotide comprising at least one polypyrimidine region, providing a parallel-stranded hairpin, said parallel-stranded hairpin comprising a purine part, a linker, and a pyrimidine part, wherein said parallel-stranded hairpin is capable of binding said target oligonucleotide, combining said target oligonucleotide and said parallel-stranded hairpin, and binding said target oligonucleotide to said parallel-stranded hairpin.

In a further aspect, the invention provides a method wherein a purine part is connected by its 5′ end to said linker, and wherein said linker is connected to the 5′ end of said pyrimidine part. In a still further aspect of the invention, said purine part comprises a modification improving stability of said parallel-stranded hairpin relative to an unmodified parallel-stranded hairpin in an identical environment of use. In yet a further aspect of the invention, the purine part comprises at least one 8-aminopurine, for example 8-aminoadenine, 8-aminoguanine, and/or 8-aminohypoxanthine.

In another aspect of the invention, the polypyrimidine region has a length of between about 9 nucleotides to between about 25 nucleotides. In a further aspect of the invention, the polypyrimidine region comprises one or two purine interruptions.

In another aspect of the invention, the parallel-stranded hairpin is capable of binding only to said target oligonucleotide. In a still further aspect of the invention, the target oligonucleotide and said parallel-stranded hairpin binding forms a triplex. In yet a further aspect of the invention, a parallel-stranded hairpin is stable at a pH range between about 4 and about 7.4. In another aspect of the invention, the target oligonucleotide and a parallel-stranded hairpin are combined in vivo. In a further aspect of the invention, a target oligonucleotide is selected from the group consisting of a virus, a bacterium, a rickettsium, a fungus, a parasite, a biological warfare agent, a dysplastic, a cancer cell, and an unwanted cell subset. In a further aspect of the invention, binding of a target oligonucleotide and a parallel-stranded hairpin has at least one effect selected from the group consisting of inhibiting DNA synthesis, inhibiting DNA transcription, and inhibiting mRNA transcription. In a further aspect of the invention, a said parallel-stranded hairpin comprises a peptide sequence. Such a peptide sequence may have a label and/or may be capable of binding a target.

In a further aspect of the invention, a target oligonucleotide is present in a biological location selected from the group consisting of tissues, cells, organs, and body fluids. In still another aspect, a parallel-stranded hairpin is provided to a patient in the presence of a non-immunogenic carrier. A non-immunogenic carrier may be a liposome. In an aspect of the invention, a parallel-stranded hairpin has no toxic effect on a patient.

In a further aspect of the invention, binding inhibits telomerase activity. In another aspect, a target oligonucleotide comprises the sequence set forth in SEQ ID NO: 19. A further aspect of the invention includes a parallel-stranded hairpin, said parallel-stranded hairpin comprising a sequence selected from the group consisting of RP8AP-Tel 16 (SEQ ID NO: 20, SEQ ID NO: 21); RP8AP-Tel 20 (SEQ ID NO: 22, SEQ ID NO: 23), and RP8AP-Tel 22 (SEQ ID NO: 24, SEQ ID NO: 25).

### FIGURES

FIG. 1 shows a number of antisense strategies.

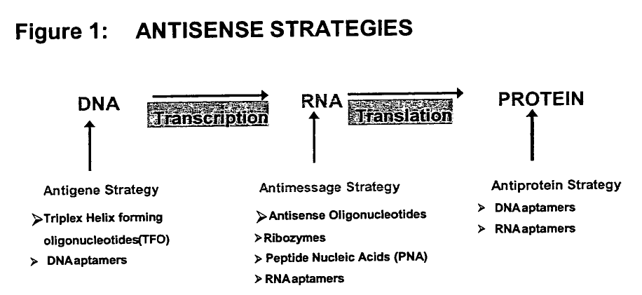

FIG. 2 shows a representation of cellular processing of genetic information.

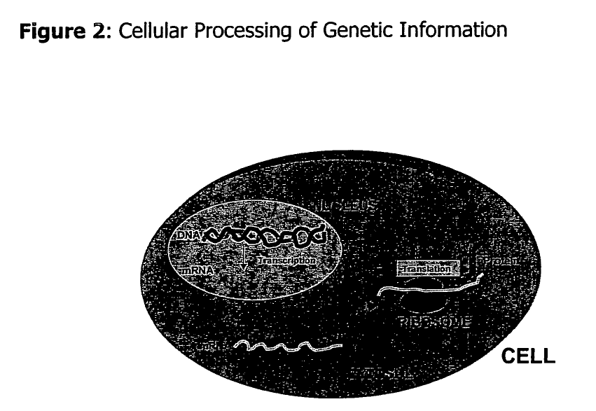

FIG. 3 shows a representation of mode of action of antisense inhibition.

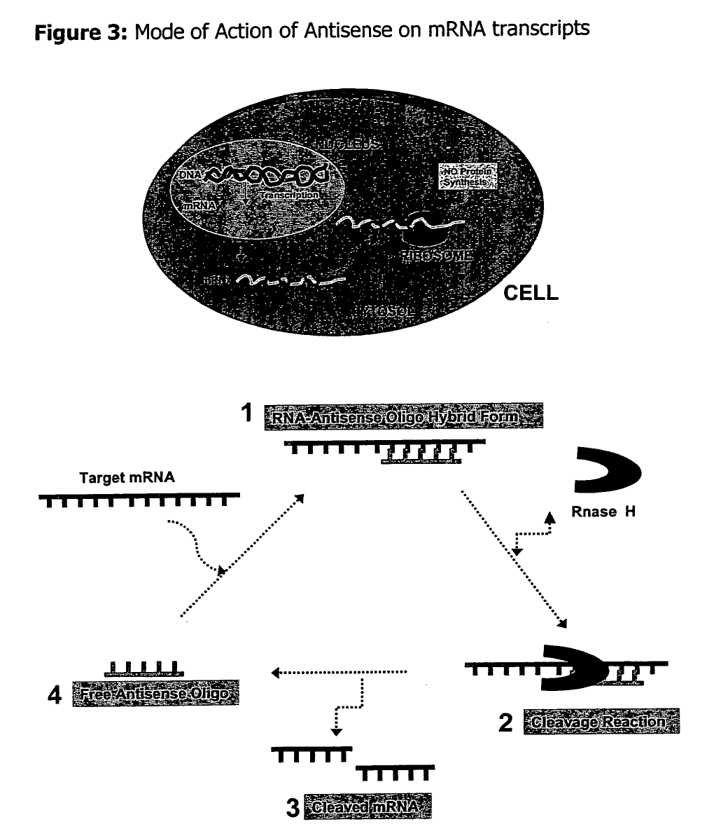

FIG. 4 shows exemplary antisense oligomers structures that may be resistant to cellular nuclease attack.

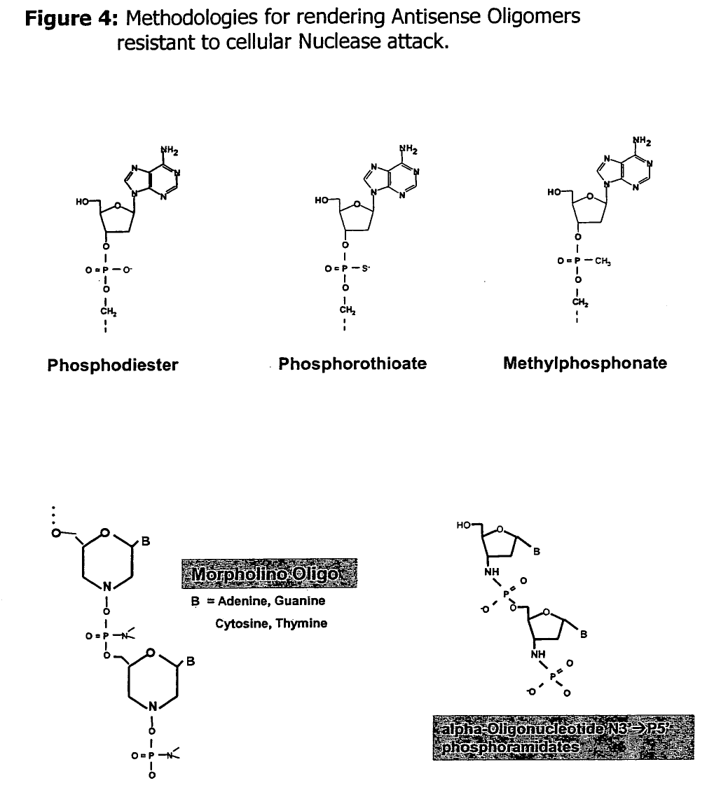

FIG. 5 shows hypothetical base-pairing schemes of triads containing 8-aminopurines.

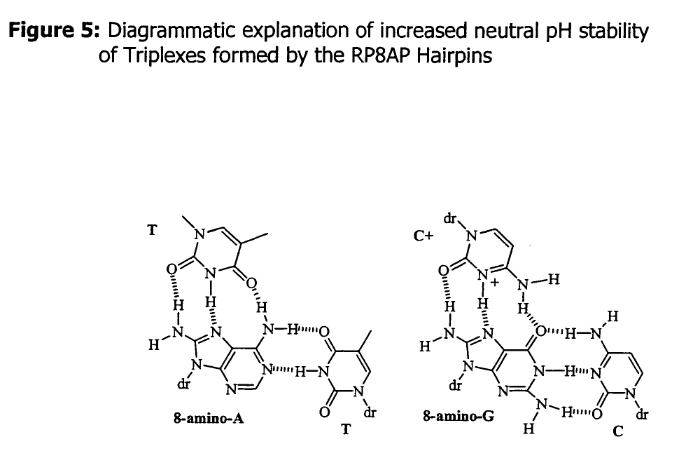

FIG. 6 shows an exemplary scheme for RP8AP Hairpin Triplex production.

FIG. 7 shows an exemplary polypyrimidine sequence found, for example, as a repeat at the end of the smallpox genome. This 20-mer (base) region will bind an RP8AP hairpin discussed herein.

Table 1 shows a number of polypyrimidine sequences found in the double stranded DNA of the smallpox viral genome.

Table 2 shows a number of exemplary polypyrimidine base sequences of greater than 20 bases in length in the smallpox viral genome. Sequences in the Watson strand have been identified in coding regions (mRNA produced) or non-coding regions (no mRNA production).

Table 3 shows a comparison of potential polypyrimidine target numbers in smallpox and cowpox viral genome sequences.

Table 4 shows sequence inspection samples of polypyrimidine target regions for the binding of exemplary RP8AP hairpins in smallpox and cowpox. Table 5 shows a human telomerase hTR (mRNA) sequence. Three exemplary RP8AP hairpins for target RNA sequences are shown.

### DESCRIPTION

The present invention is directed to methods and compositions of nucleic acid structures that may be used as antisense oligonucleotides for selected target molecules. An aspect of the invention includes compositions and methods for the preparation of oligonucleotides including modified nucleotides, for example but not limited to 8-aminoadenine, 8-aminoguanine, 8-aminohypoxanthine, among others, that are connected 3′ to 3′ or 5′ to 5′ (head-to-head or tail-to tail) to a Hoogsteen pyrimidine strand (parallel-stranded hairpins).

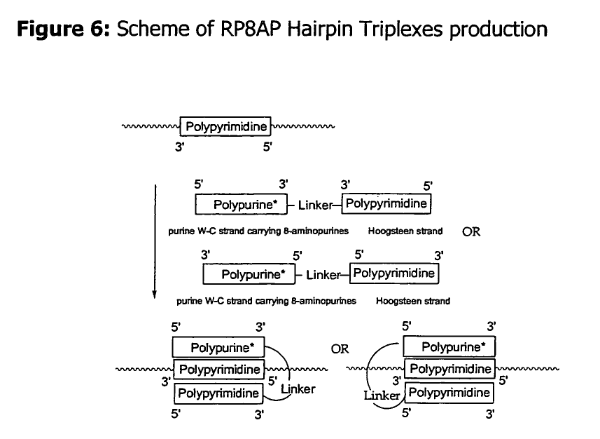

In one aspect, compositions of the invention include parallel-stranded oligomers comprising at least one 8-aminopurine. A further aspect of the invention comprises oligonucleotide derivatives comprising two parts, a polypyrimidine part connected head-to-head to a complementary purine part carrying one or more 8-aminopurines, for example 8-aminoadenine, 8-aminoguanine, and/or 8-aminohypoxanthine. In a further aspect of the invention, a linker molecule is located between both parts in such a way that both parts can form a double stranded structure in parallel sense (FIG. 6). In a further aspect of the invention, a polypyrimidine part is connected tail-to-tail to a complementary purine part carrying one ore more 8-aminopurines. A method for synthesizing the oligonucleotides of the present invention includes use of phosphoramidite chemistry. Oligonucleotides may be synthesized by any method known to those skilled in the art. The sequences of the parallel-stranded hairpins may be defined and identified. They could be as long as about 40 bases (about 20 normal polarity and about 20 reversed polarity). Parallel-stranded hairpins and/or triplexes may carry a peptide sequence that may be used as non-radioactive label, or the peptide may recognize and binds to a specific cell receptor, protein, or other kind of molecule target.

Designed parallel-stranded hairpins may have a sequence capable of acting as a nucleic acid antisense or antigene oligonucleotide to a desired target molecule. Moreover, they may be able to discriminate between closely related molecules. Parallel-stranded hairpins may contain modified nucleotides or other modifications to increase characteristics such as resistance to intra-cellular and extra-cellular nucleases or in vivo stability.

Stability of a triplex helix at broad pH (for example, between about 4 to about 7.6) conditions using RP8AP hairpins, and the possibility to cope with, for example, one or two purine base interruptions in the polypyrimidine target sequence offers great potential for their application, based on triplex helix formation, to antigene and antisense therapies. An attractive target for RP8AP includes telomerase. As previously stated, a hairpin may be designed to form a triplex with a single RNA strand region. The telomerase enzyme is known to possess a region called hTR, which is a single strand RNA. Several RP8AP potential hairpin-binding regions exist on the hTR region. These polypyrimidine regions include the following positions of the hTR region: base 130 to 142 (13-mer) and base 190 to 203 (14-mer). These and/or any other suitable sequence in the hTR region (RNA) can be inactivated by the binding with a selected RP8AP hairpin. Inhibition of this telomerase activity by binding with a hairpin may hinder or stop cancer cell division and enable the body of a patient to resolve the pathologic cells.

### EXAMPLE I

### Therapeutic Strategies to Block the Variola (Smallpox) and Vaccinia Virus Infection Disease Course

Due to the virulence and contagiousness of smallpox virus, its laboratory investigation has been limited. The closely related and much less dangerous vaccinia virus is the model of choice for smallpox study. This example and the others included in this application are prophetic and have not been performed.

Viral particles from smallpox-infected individuals are indistinguishable from vaccinia particles. They are characterized as a central nucleoid with a dumbbell shaped dense coil composed of viral DNA. The nucleoid is surrounded by lipoprotein membranes and between the nucleoid and the outer viral coat is an ellipsoidal body. The viral cores contain viral DNA as well as several enzymes, primarily for transcription and modification of intermediate early mRNAs including, for example, DNA dependent RNA polymerase, polyadenylate polymerase, methyltransferase, and guanyltransferase.

RP8AP hairpins are used as antisense oligonucleotides to inhibit replication of the smallpox virion. These hairpins are designed, for example, to inhibit viral DNA replication, to inhibit mRNA transcript synthesis, and to inhibit mRNA translation. The immediate early mRNAs produced by the viral enzyme, DNA dependent RNA polymerase, found in the viral core along with viral DNA are the earliest targets for binding of the DNA hairpins with subsequent formation of a stable RNA.DNA.DNA triplex where the RNA is the messenger RNA produced.

The RP8AP hairpin is designed to bind to a polypyrimidine region of 9 to 25 nucleotides with one or two purine interruptions. Any of the mRNA monocystronic messages that possess such a sequence may be a target of the RP8AP hairpin. Accordingly, a hairpin is designed to bind the polypyrimidine region on the mRNA transcripts produced. Complexation in vivo of the specific mRNA transcript and its binding hairpin result in inhibition of mRNA translation by the formation of a stable triplex, rendering the mRNA transcript incapable of producing viral protein used to progress the viral infection.

The smallpox virus produces about 80 distinct species of polypeptide each with a unique mRNA transcript. Each mRNA transcript can be targeted by an appropriate RP8AP hairpin. Again, inhibition of production of any single polypeptide may stop viral particle production and maturation and prevent the formation of virulent mature virions. Herein the hairpin is used as an antisense oligonucleotide to block translation of a viral mRNA transcript.

A second strategy employing binding of the hairpin includes non-coding DNA regions on the Watson or Crick stand, for example those in controlling or promoter regions may inhibit viral DNA synthesis. Table I shows about 5,000,12-mer regions on the Watson strand that may be utilized as targets for the RP8AP hairpin. A similar number of targets (about 5,000) for the hairpin also exist on the Crick strand. These exemplary DNA targets on either strand may be utilized to block viral DNA replication. Viral DNA replication may be more effectively blocked if the triplex forms in a DNA control or promoter region. A single hairpin forming a triplex in a replication fork throughout the viral genome may confer a similar effect.

Table II shows eighteen exemplary polypyrimidine sequences of greater than about 20-mer that exist in a smallpox genome. Each is capable of being targeted by the hairpins. Seven exist in coding regions of DNA, being found in the mRNA transcripts, while 11 identical sequences exist in the non-coding region found clustered at the end of the smallpox genome. These identical sequences are separated by 70-mer base sequence. This region may represent the promoter region for DNA synthesis as well as the controlling region for initiation of transcription. This may be considered a prime target for inactivation of the smallpox viral genome.

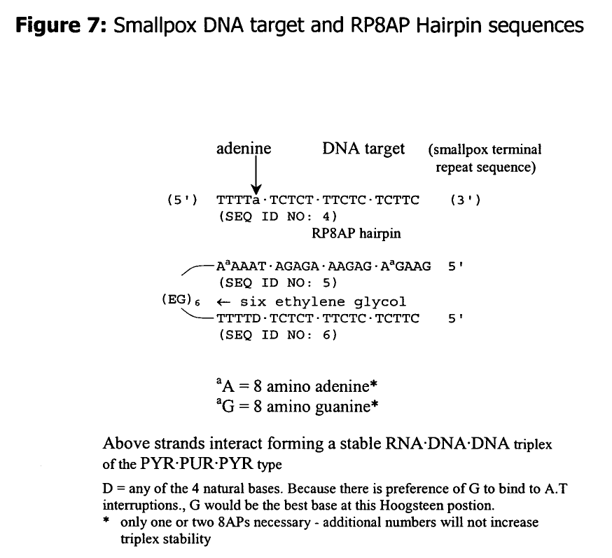

FIG. 7 shows the sequence of a 20-mer target with a single mismatch that is identically repeated 11 times and separated by 70-mer regions. A hairpin sequence specific for each of the 11 repeats may be constructed to form a stable triplex, inhibiting activity of this viral controlling region at the end of the viral genome.

One 1 5-mer polypyrimidine target found in the variola virus genome is represented as (5′)TCTCTTTCTCTCTTC(3′) (SEQ ID NO: 1) and the structure of the binding hairpin is represented as (5′) CTTCTCTCTTTCTCT (3′)-(EG)₆-(3′)AGAGAAAGAGAGAAG (5′) (SEQ ID NO: 2, SEQ ID NO: 3), wherein two guanines in the polypurine region are 8-amino substituted Gs, which provide high stability to the triplex at physiologic pH.

Table 3 shows a comparison of potential polypyrimidine target numbers between Smallpox and Cowpox genomes. Some of those targets for binding of RP8AP are shown on Table 4.

Biosynthesis of the components of these viruses and their assembly into viral particles take place entirely within the cytoplasm of the cell. Most biosynthetic reactions can occur in enucleated cells, however, a large subunit of the nuclear host RNA polymerase II joins with a virus encoded RNA polymerase subunit to transcribe a unique set of special mRNAs. Virus attaches to uncharacterized host cell receptors and by engulfment enters the cytoplasm. After penetration, viral DNA is released by a two-stage uncoating process.

Stage I uncoating is initiated immediately after engulfment by preexisting host cell enzymes which breakdown the viral membrane part of the protein coat of the viral particle and the membrane of the endocytic vesicle to release the nucleoprotein core into the cytoplasm.

Stage II of the uncoating process results in core breakdown to liberate viral DNA. At the onset of this stage a DNA-dependent RNA polymerase, present in the intact core, transcribes about 25% of the viral genome. These resulting transcripts are processed within the core and functional immediately as early mRNAs emerge, which encode for proteins required for the final uncoating events and for the enzymes necessary to produce the second set of mRNAs, the delayed early. After viral DNA replication begins, late mRNAs appear that are derived from about 60% of the genome, while synthesis of early mRNAs continue.

These mRNA species may present appropriate binding sites for hairpins introduced into a cell. Binding would result in stable triplex formation in from acidic to physiologic pH. Transcription continues until about 7 hours after infection.

Synthesis of specific enzymes and of a few viral structural proteins begins early in biosynthesis before replication of viral DNA. These proteins include second-stage uncoating proteins, three proteins associated with the nucleoprotein core, a protein essential for initiation of viral DNA replication and enzymes related to DNA synthesis. Viral DNA begins to be synthesized about 1.5 to about 2 hours after infection and achieves maximal concentration by the time of detection of newly made infectious virus. DNA replication involves a unique covalent cross-linking of the two strands. Synthesis is initiated at either end of the genome. Large circular and forked replicating forms are found indicating that an endonuclease cleaves the single-stranded cross-links during replication.

In a further aspect of the invention, the RP hairpins may target an appropriate site in viral DNA to form a DNA.DNA.DNA triplex in the area of, for example, the replication forks. Stable triplex formation may inhibit a number of enzymatic reactions, including endonuclease, DNA polymerase and RNA polymerase activities. In vivo triplex formation in exposed single strand areas within the replication fork may irreversibly block viral DNA synthesis. This triplex formation would be highly stable in the physiologic pH of the cellular cytoplasm.

Late viral proteins are first detected about 4 hours post infection and infectious virus is formed about 1 hour later by packaging viral DNA randomly selected from the preformed pools. Post translational modifications of several proteins (cleavage, glycosylation, and phosphorylation) are essential to virion maturation: Host cell macromolecule synthesis is inhibited during mRNA and DNA synthesis of the virus. Production of host protein steps due to blockage of initiation of peptide chain synthesis of host proteins and host cell polyribosomes are disrupted, host DNA ceases replication, and the host mRNAs cannot leave the nucleus. It is unknown how the virus exerts its control on host cell macromolecule synthesis. As viral DNA synthesis increases, regions of dense fibrous material appear in the cell cytoplasm. About 3 hours post-infection, some of the early proteins form membrane like structures, which begin to enclose patches of viral components and proceed to form immature particles into which the DNA enters. Upon completion of the viral envelope, the nucleoid begins to form within the immature particle. An additional membrane encloses the condensing DNA, the lateral bodies differentiate and finally the outer coat structures are laid down on the previously formed membrane, thereby completing the assembly of mature virions. These infectious virions are released by a double mechanism, one, release through cell villi, and two, release by lysis of the cell.

The smallpox virus is said to be transmitted by droplet infection. Initial lesions develop in the upper respiratory tract. Dissemination by foamites is important due to the fact that the virus is resistant to ordinary temperatures and drying. Airborne transmission may occur. Viral particles released from the initial lung lesions travel via the bloodstream to the epidermal skin cells preferred for smallpox viral replication.

Two basic forms of smallpox are known: variola major (fatality rate of about 25%), and variola minor, or alastrim, a less virulent form (fatality rate below about 1%). The viruses are generally indistinguishable. Virus multiplies initially in the mucose of the upper respiratory tract and next in regional lymph nodes. A transient viremia allows dissemination of the virus to internal organs (liver, spleen, and lungs) where the virus propagates extensively. A second viral invasion of the bloodstream terminates the incubation period (about 12 days) and initiates the toxemic phase, characterized by prodromal macular rashes, generalized aching, headache, malaise, and prostration. Virus spreads to the skin and multiplies in the epidermal cells. The characteristic skin eruptions follow in about 3 to about 4 days. Macular at onset, the rash progresses from papular to vesicular, and finally pustular in severe cases the rash may become hemorrhagic or confluent. Inclusion bodies, guarnieri bodies, characteristically develop in the cells of the skin and mucous membranes infected with variola or vaccinia. Each inclusion body consists of an accumulation of viral particles and viral antigens (observed in all other poxvirus infections). A hypersensitivity response to the viral antigens may contribute to the eruptive lesions and the toxin like properties of the viral particles may also play a role in cell necrosis.

One site to fight viral infection is at the site of infection in the lungs. Hairpins may be enclosed in liposomes, which may be aerosolized and introduced into the lungs. Limitations to this delivery strategy focus on the knowledge that the individual has suffered exposure to the virus. It is believed that the liposome aerosol could provide short-term prophylactic immunity to individuals at risk, for example, military personnel. Long-term viral particle elimination strategies would involve a technology known as Selected Target Elimination (STE I/STE II), as reported in United States Published Patent Application No. 2003-0232045 A1, in the name of Ramberg, et al.

Initial disease manifestations develop as the virus travels from the lungs to the epidermal cells of the skin, a favored site for viral replication. At this stage, hairpins may be added into liposomes and directly applied to erupting skin lesions. At this point lung inhalation of the hairpin-loaded liposomes would stem the flow of virions into the bloodstream for dissemination into the body. Liposomes loaded with RP8AP hairpins may be introduced intravenously and protect organs from infection.

### EXAMPLE II

### Therapeutic Strategies to Block Cancer Progression and Assist in its Resolution

Cancer cells depend on a functional cellular telomerase enzyme activity. Inhibition of telomerase activity may block progression of a number of types of cancer. Table 5 shows a sequence of an hTR or RNA (single strand) part of the telomerase enzyme. The two underlined sequences are exemplary polypyrimidine regions that can be targeted by the RP8AP hairpins. Herein are presented three hairpin sequences, and their target region on the hTR region are delineated. Administration of topical creams containing hairpin filled liposomes to combat melanomas and other skin cancers may resolve the cancer state.

Currently cryosurgical conization is used to treat a women suspected of having cervical atypia or carcinoma in situ. This procedure often must be repeated due because the dysplastic site has not been directly attacked. The telomerase hairpin may provide a functionality to inhibit rapidly growing cells in a body region typically containing dead and dying exfoliative cells. A hairpin-loaded liposome may, in a suppository format, directly involve the pathologic cells while providing little or no toxicity to normal cells found in the region.

Use of the hairpin as an anti-cancer drug should be considered valuable as long as toxicity to normal cells have been taken into consideration and the cancer cell is in a body area devoid of rapidly growing cells.

Patents, patent applications, publications, scientific articles, books, web sites, and other documents and materials referenced or mentioned herein are indicative of the levels of skill of those skilled in the art to which the inventions pertain. Each such referenced document and material is hereby incorporated by reference to the same extent as if it had been incorporated by reference in its entirety individually or set forth or reprinted herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, and the claims referred to above including but not limited to any original claims.

The inventions have been described broadly and generically herein. Each of the narrower species and subgeneric groupings falling within the generic disclosure also form part of these inventions. This includes the generic description of each invention which hereby include, including any claims thereto, a proviso or negative limitation removing or optionally allowing the removal of any subject matter from the genus, regardless of whether or not the excised materials or options were specifically recited or identified in haec verba herein, and all such variations form a part of the original written description of the inventions. In addition, where features or aspects of an invention are described in terms of a Markush group, the invention shall be understood thereby to be described in terms of each and every, and any, individual member or subgroup of members of the Markush group.

The inventions illustratively described and claimed herein can suitably be practiced in the absence of any element or elements, limitation or limitations, not specifically disclosed herein or described herein as essential. Thus, for example, the terms “comprising,” “including,” “containing,” “for example”, etc., shall be read expansively and without limitation. In claiming their inventions, the inventors reserve the right to substitute any transitional phrase with any other transitional phrase, and the inventions shall be understood to include such substituted transitions and form part of the original written description of the inventions. Thus, for example, the term “comprising” may be replaced with either of the transitional phrases “consisting essentially of” or “consisting of.”

As used herein and in the appended claims, the singular forms “a,” “an,” and “the” include plural reference unless the context clearly dictates otherwise.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement was specifically and without qualification or reservation expressly adopted by Applicants in a responsive writing specifically relating to the application that led to this patent prior to its issuance.

The terms and expressions employed herein have been used as terms of description and not of limitation, and there is no intention in the use of such terms and expressions, or any portions thereof, to exclude any equivalents now know or later developed, whether or not such equivalents are set forth or shown or described herein or whether or not such equivalents are viewed as predictable, but it is recognized that various modifications are within the scope of the invention claimed, whether or not those claims issued with or without alteration or amendment for any reason. Thus, it shall be understood that, although the present invention has been specifically disclosed by preferred embodiments and optional features, modifications and variations of the inventions embodied therein or herein disclosed can be resorted to by those skilled in the art, and such modifications and variations are considered to be within the scope of the inventions disclosed and claimed herein.

Specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. Where examples are given, the description shall be construed to include but not to be limited to only those examples. It will be readily apparent to one skilled in the art that varying substitutions and modifications may be made to the invention disclosed herein without departing from the scope and spirit of the invention, and from the description of the inventions, including those illustratively set forth herein, it is manifest that various modifications and equivalents can be used to implement the concepts of the present invention without departing from its scope. A person of ordinary skill in the art will recognize that changes can be made in form and detail without departing from the spirit and the scope of the invention. The described embodiments are to be considered in all respects as illustrative and not restrictive. Thus, for example, additional embodiments are within the scope of the invention and within the following claims. In the following claims, where the phrase “providing a” is used, it is to be construed to mean both providing a single and providing a plurality of the object.

TABLE 1
Variola (Smallpox) Genome Polynucleotide Regions (Entire
DNA) Based On Genebank Sequence

| | | |
|---|---|---|
| MER | Interruptions | Total Number Sequences |

POLYPYRIMIDINE IN WATSON STRAND

| | | |
|---|---|---|
| 12 | 2 | 3,946 |
| 12 | 1 | 838 |

POLYPURINE IN WATSON STRAND OR POLYPYRIMIDINE
IN CRICK STRAND

| | | |
|---|---|---|
| 12 | 2 | 3,949 |
| 12 | 1 | 816 |

TABLE 2
Variola (Smallpox) Genome Polynucleotide Regions (Coding
And Non-Coding Based On Genebank Sequence

| | | |
|---|---|---|
| MER | Interruptions | Total Number Sequences |

POLYPYRIMIDINE IN WATSON STRAND

| | | |
|---|---|---|
| >20 | 1 | 18* |

POLYPURINE IN WATSON STRAND OR POLYPYRIMIDINE
IN CRICK STRAND

| | | |
|---|---|---|
| <20 | 1 | 6 |

*Coding region (mRNA) 7; Non-coding region (DNA) 11**
**Identical sequences separated by 70 bases exactly found in cluster at the end of the smallpox genome

TABLE 3
Comparison of potential polypyrimidine target numbers in
Smallpox and Cowpox
GeneBank Accession # NC_001611 (Variola) and NC_003663
(Cowpox), complete genomes

| | Smallpox | Cowpox | DNA Strand |
|---|---|---|---|
| Total BP (base pairs) | 185578 | 224501 | |
| #12 BP polyPur w/0 Pyr | 126 | 267 | WATSON |
| #12 BP polyPur w/1 Pyr | 838 | 1277 | |
| #12 BP polyPur w/2 Pyr | 3946 | 5182 | |
| #12 BP polyPur w/0 Pyr | 78 | 275 | CRICK |
| #12 BP polyPur w/1 Pyr | 816 | 1307 | |
| #12 BP polyPur w/2 Pyr | 3949 | 5118 | |

TABLE 4

Samples of Polypyrimidine target regions for
the binding of the RP8AP Hairpins in
Smallpox and Cowpox

| | | |
|---|---|---|
| Base # | Gene | Sequence (5′ - 3′) |

Smallpox

| | | |
|---|---|---|
| 5401 | D6L | CaTCTCCCCCTTTCTTTTTT |
| | | (SEQ.. ID NO. 7) |
| 26166 | C11L | TaTCCTTCTCCTTCCTCTTCT |
| | | (SEQ. ID NO 8) |
| 53917 | K4L | TaTCTCTTTTCTCTTTC |
| | | (SEQ. ID NO. 9) |
| 64932 | H7L | TTTTTTCCCTCgTTCTTTTTCTT |
| | | (SEQ. ID NO 10) |
| 125303 | A24R | TCTCTCTCCTCTCTT |
| | | (SEQ. ID NO. 11) |
| 137431 | A40_5R | TTCaTTCTCTTCTCTTTTT |
| | | (SEQ. ID NO. 12) |
| 184738 | TR* | TTTTaTCTCTTTCTCTCTTC × 11 repeats |
| | | (SEQ ID NO: 13) |

Cowpox

| | | |
|---|---|---|
| 27968 | V024 | CaTCTCCCCCTTCCTTTTTT |
| | | (SEQ ID NO: 14) |
| 90848 | V092 | TTTTTTCCCTCgTTCTTTTTCTT |
| | | (SEQ ID NO: 15) |
| 163767 | V167 | TTCaTTCTTCTCTTCTCTTTTT |
| | | (SEQ ID NO: 16) |
| 193941 | NCR** | TTTCTTCTTTCCTCCCTCTTaTCCCTTTCCC |
| | | (SEQ ID NO: 17) |
| 21994 | TR* | TTTTTaTTCTCTTTCTCTCTTC × 31 repeats |
| | | (SEQ ID NO: 18) |

*TR = 3′ Terminal Repeat sequence - Base # indicates position of 1^{st} repeat. This is a non-coding region.

TABLE 5
Human Telomerase (hTR or RNA) sequence

| | |
|---|---|
| Base | |
| Count | /gene = “hTR” |
| Origin | 81 a 182 c 185 g 97 t |

| | |
|---|---|
| (5′)1 | gagtgactct cacgagagcc gcgagagtca gcttggccaa tccgtgcggt cggcggccgc |
| 61 | tccctttata agccgactcg cccggcagcg caccgggttg cggagggtgg gcctgggagg |
| 121 | ggtggtggcc attttttgtc taaccctaac tgagaagggc gtaggcgccg tgcttttgct |
| 181 | ccccgcgcgc tgtttttctc gctgactttc agcgggcgga aaagcctcgg cctgccgcct |
| 241 | tccaccgttc attctagagc aaacaaaaaa tgtcagctgc tggcccgttc gcccctcccg |
| 301 | gggacctgcg gcgggtcgcc tgcccagccc ccgaaccccg cctggaggcc gcggtcggcc |
| 361 | cggggcttct ccggaggcac ccactgccac cgcgaagagt tgggctctgt cagccgcggg |
| 421 | tctctcgggg gcgagggcga ggttcaggcc tttcaggccg caggaagagg aacggagcga |
| 481 | gtccccgcgc gcggcgcgat tccctgagct gtgggacgtg cacccaggac tcggctcaca |
| 541 | catgc (3′) (SEQ ID NO: 19) |

Revised: Oct. 24, 2001. NCBI Sequence Viewer
hTR sequence Base 129 to Base 141
Corresponding RP8AP hairpin

| | | | | | |
|---|---|---|---|---|---|
| RP8AP- | 5′ ctc ttt tt-3′ | (EG) 6-3′ | aaaaaGag 5′ | G = 8-aminog | 16 mer |
| Tel 16 | (SEQ ID NO: 20) | | (SEQ ID NO: 21) | | |

hTR sequence Base 190 to Base 203
Corresponding RP8AP_hairpin

| | | | | | |
|---|---|---|---|---|---|
| RP8AP- | 5′ tctctttttt-3′ | (EG) 6-3′ | aaaaaacaGa 5′ | G = 8-aminog | 20 mer |
| Tel 20 | 1 (SEQ ID NO: 22) | | (SEQ ID NO: 23) | | |
| RP8AP- | 5′ tccctcttttt-3′ | (EG) 6-3′ | aaaaaGagcga 5′ | G = 8-aminog | 22 mer |
| Tel 22 | (SEQ ID NO: 24) | | (SEQ ID NO: 25) | | |

1. A method for binding a target oligonucleotide, comprising:
providing a target oligonucleotide, said target oligonucleotide comprising at least one polypyrimidine region;
providing a parallel-stranded hairpin, said parallel-stranded hairpin comprising a purine part, a linker, and a pyrimidine part, wherein said parallel-stranded hairpin is capable of binding said target oligonucleotide;
combining said target oligonucleotide and said parallel-stranded hairpin; and
binding said target oligonucleotide to said parallel-stranded hairpin.

2. The method of claim 1, wherein said purine part is connected by its 5′ end to said linker, and wherein said linker is connected to the 5′ end of said pyrimidine part.

3. The method of claim 2, wherein said purine part comprises a modification improving stability of said parallel-stranded hairpin relative to an unmodified parallel-stranded hairpin in an identical environment of use.

4. The method of claim 2, wherein said purine part comprises at least one 8-aminopurine.

5. The method of claim 4, wherein said 8-aminopurine is selected from the group consisting of 8-aminoadenine, 8-aminoguanine, and 8-aminohypoxanthine.

6. The method of claim 1, wherein said polypyrimidine region has a length of between about 9 nucleotides to between about 25 nucleotides.

7. The method of claim 6, wherein said polypyrimidine region comprises one or two purine interruptions.

8. The method of claim 1, wherein said parallel-stranded hairpin is capable of binding only to said target oligonucleotide.

9. The method of claim 1, wherein said target oligonucleotide and said parallel-stranded hairpin binding forms a triplex.

10. The method of claim 1, wherein said parallel-stranded hairpin is stable at a pH range between about 4 and about 7.4.

11. The method of claim 1, wherein said combining occurs in vivo.

12. The method of claim 1, wherein said target oligonucleotide is selected from the group consisting of a virus, a bacterium, a rickettsium, a fungus, a parasite, a biological warfare agent, a dysplastic, a cancer cell, and an unwanted cell subset.

13. The method of claim 1, wherein said binding has at least one effect selected from the group consisting of inhibiting DNA synthesis, inhibiting DNA transcription, and inhibiting mRNA transcription.

14. The method of claim 1, wherein said parallel-stranded hairpin comprises a peptide sequence.

15. The method of claim 14, wherein said peptide sequence has a label.

16. The method of claim 14, wherein said peptide is capable of binding a target.

17. The method of claim 1, wherein said target oligonucleotide is present in a biological location selected from the group consisting of tissues, cells, organs, and body fluids.

18. The method of claim 1, wherein said parallel-stranded hairpin is provided to a patient in the presence of a non-immunogenic carrier.

19. The method of claim 18, wherein said non-immunogenic carrier is a liposome.

20. The method of claim 1, wherein said binding inhibits telomerase activity.

21. The method of claim 1, wherein said target oligonucleotide comprises the sequence set forth in SEQ ID NO: 19.

22. The method of claim 11, wherein said parallel-stranded hairpin has no toxic effect on a patient.

23. A parallel-stranded hairpin, said parallel-stranded hairpin comprising a sequence selected from the group consisting of RP8AP-Tel 16 (SEQ ID NO: 20, SEQ ID NO: 21); RP8AP-Tel 20 (SEQ ID NO: 22, SEQ ID NO: 23), and RP8AP-Tel 22 (SEQ ID NO: 24, SEQ ID NO: 25).


SEQUENCE LISTING (25 sequences)

SEQ ID NO: 1 - DNA, 15 bp, Variola virus
tctctttctctcttc

SEQ ID NO: 2 - DNA, 15 bp, Artificial
  Pyrimidine portion of hairpin
cttctctctttctct

SEQ ID NO: 3 - DNA, 15 bp, Artificial
  Modified Polypurine region of hairpin Optimally 2 8-aminoguanines in place of guanines in the sequence
gaagagagaaagaga

SEQ ID NO: 4 - DNA, 20 bp, Variola virus
ttttatctctttctctcttc

SEQ ID NO: 5 - DNA, 20 bp, Artificial Sequence
  Hairpin Component
gaanagagaaagagataana

SEQ ID NO: 6 - DNA, 20 bp, Artificial Sequence
  Hairpin Component′
cttctctctttctctntttt

SEQ ID NO: 7 - DNA, 20 bp, Variola virus
catctccccctttctttttt

SEQ ID NO: 8 - DNA, 21 bp, Variola virus
tatccttctccttcctcttct

SEQ ID NO: 9 - DNA, 17 bp, Variola virus
tatctcttttctctttc

SEQ ID NO: 10 - DNA, 23 bp, Variola virus
ttttttccctcgttctttttctt

SEQ ID NO: 11 - DNA, 15 bp, Variola virus
tctctctcctctctt

SEQ ID NO: 12 - DNA, 19 bp, Variola virus
ttcattctcttctcttttt

SEQ ID NO: 13 - DNA, 20 bp, Variola virus
ttttatctctttctctcttc

SEQ ID NO: 14 - DNA, 20 bp, Cowpox virus
catctcccccttcctttttt

SEQ ID NO: 15 - DNA, 23 bp, Cowpox virus
ttttttccctcgttctttttctt

SEQ ID NO: 16 - DNA, 22 bp, Cowpox virus
ttcattcttctcttctcttttt

SEQ ID NO: 17 - DNA, 31 bp, Cowpox virus
tttcttctttcctccctcttatccctttccc

SEQ ID NO: 18 - DNA, 22 bp, Cowpox virus
tttttattctctttctctcttc

SEQ ID NO: 19 - DNA, 545 bp, Homo sapiens
gagtgactctcacgagagccgcgagagtcagcttggccaatccgtgcggtcggcggccgctccctttataagccgactcgcccggcagcgcaccgggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgc

SEQ ID NO: 20 - DNA, 8 bp, Artificial Sequence
  Hairpin Component for RP8AP Hairpin
ctcttttt

SEQ ID NO: 21 - DNA, 8 bp, Artificial Sequence
  Hairpin Component 2 for RP8AP Hairpin
ganaaaaa

SEQ ID NO: 22 - DNA, 10 bp, Artificial Sequence
  Hairpin Component
tctctttttt

SEQ ID NO: 23 - DNA, 10 bp, Artificial Sequence
  Hairpin component
anacaaaaaa

SEQ ID NO: 24 - DNA, 11 bp, Artificial Sequence
  Hairpin component
tccctcttttt

SEQ ID NO: 25 - DNA, 11 bp, Artificial Sequence
  Hairpin component
agcganaaaaa